(19) United States
(12) Patent Application Publication
(10) Pub. No.: US 2023/0276773 A1
(43) Pub. Date: Sep. 7, 2023

(54) SYSTEMS AND METHODS FOR AUTOMATIC AND NONINVASIVE LIVESTOCK HEALTH ANALYSIS

(71) Applicant: BOARD OF TRUSTEES OF MICHIGAN STATE UNIVERSITY, East Lansing, MI (US)

(72) Inventors: Madonna BENJAMIN, East Lansing, MI (US); Michael LAVAGNINO, Lansing, MI (US); Steven YIK, East Lansing, MI (US); Daniel MORRIS, Okemos, MI (US)

(21) Appl. No.: 17/926,916

(22) PCT Filed: May 21, 2021
(86) PCT No.: PCT/US2021/033744

§ 371 Date: Nov. 21, 2022

Related U.S. Application Data
(60) Provisional application No. 63/028,507, filed on May 21, 2020.

(51) Int. Cl. A01K 29/00; G06V 40/10; G06V 10/82; G06T 7/579

(52) U.S. Cl. CPC A01K 29/005; G06V 40/10; G06V 10/82; G06T 7/579; G06T 2207/10016; G06T 2207/10028; G06T 2207/10048; G06T 2207/20081; G06T 2207/20084

(57) ABSTRACT

The disclosure provides systems and methods for automatically and noninvasively analyzing livestock health, wherein to determine at least one of a body composition indicator or a pose indicator based on the data acquired from the camera; store the body composition indicator or pose indicator in a data record associated with the animal of interest; and provide the body composition indicator or pose indicator to a neural network trained to predict an animal outcome for animals of a similar species to the animal of interest.

### CROSS-REFERENCE TO RELATED APPLICATIONS

This application claims priority to and the benefit of provisional patent application Ser. Nos. 63/028,507 filed in the United States Patent and Trademark Office (USPTO) on May 21, 2020, the entire content of which is incorporated herein by reference as if fully set forth below in its entirety and for all applicable purposes.

### FIELD

The present disclosure relates generally to the field of livestock farming. More particularly, various embodiments and advantages described below relate to systems and methods for monitoring and assessing health characteristics of livestock in precision livestock farming applications.

### BACKGROUND

Pork is the most consumed animal protein (108.2 metric tons/per year), and as global populations climb along with disposable income, a competitive race has come about to meet this demand. The largest consumers of pork are affected by the loss of pork production due to African Swine Fever. The United States is well positioned to meet these demands with an inventory of 77.7 million head, up 3% from June 2019. Subsequently, US Hog Futures pricing have climbed from $50.00/cwt to $90.00/cwt. If feedstuffs remain stable, US pork producers will gain profits and sow retention will expand. Over 12 million sows are expected to farrow in 2019, up 2% from 2018. Efficient and prosperous pork production starts with the productivity of the sow (female or mother pig) which can have the capability of producing 22 weaned piglets per year for gross revenue of $771.00 per sow/year (22 weaners*$35.05). Thin sows, however, tend to have poor reproductive performance, and may render a lower price per pound and/or the animal may be condemned with no return to the farmer.

Loss of reproductive performance is commonly a result of abnormal body condition and lameness. Fat sows tend to wean fewer piglets, which may be due to an increase in piglet mortality caused by crushing. Lameness, another welfare concern, is also associated with reduced sow longevity and productivity. Taken together, loss of productivity against feed costs, housing, and potential gains from pig sales are estimated by one source to be between $57.00 for loss of weaned pig sales up to $300.00 if the sow and her litter dies near parturition.

It is important for pig producers to maximize reproductive potential during sows' lifetime in order to decrease production costs. Sows have the capability of producing 10-12 weaned piglets per litter and if she stays in the herd for more than 4 litters, a sow would produce upwards of 40 piglets per sow lifetime. United States Pig Analytics data show that a sow death rate is about 12.2% and culling is about 42%, resulting in herd replacement rates of 50% or more. Culling decisions by farmers are made based on reproductive performance, often as a result of abnormal body condition and lameness caused by locomotion disorders. Thin sows tend to have poor reproductive performance and render a lower cull price per pound and fat sows tend to wean fewer piglets, which may be due to an increase in piglet mortality caused by crushing. Poor locomotion due to lameness, another welfare concern, is also associated with reduced sow longevity and productivity and losses of between $57 to $300/sow.

Sows have a return on investment at about 4 litters, an average of 2.2 litters per year and typically wean 10-12 pigs/litter. However, sows that are fat, wean an average of 0.74 piglets less per litter, thought to be due to increased crushing of piglets. Alternatively, preliminary data on 900 sows, demonstrates that thin sows have abnormal weaning to mating intervals.

Nutrition may represent about 60% of total production costs in raising pigs. Farms estimate that reduced overfeeding of sows improves profits by $12.00/sow/year, yet sows need to have adequate body weight and condition after weaning their piglets to avoid being culled for failure to breed back As noted, sows are most often culled due to poor body composition and locomotion. Sow cull prices increase per cwt with an increase in body weight. Cull sows in the lighter weight category (less than 450 lbs.) could profitably be fed to the next weight class. Based on Nov. 1, 2019 USDA pricing, feeding a cull sow weighing 400 lbs. for an additional 2 to 4 weeks prior to slaughter could result in an increase of $44.70 per sow sent to slaughter. However, once transported from the farm, sows pass through a complex marketing chain which involves numerous collection points which can exacerbate weight loss and lameness.

To maintain a consistent flow of breeding females and reduce economic inefficiencies, lost or culled sows are replaced with pre-ordered and schedule delivery of gilts. The arrival of breeding stock presses employees to predict and decide on which sows should be culled to make room for the incoming gilts. Yet the industry lacks quantitative, non-invasive methods of animal assessment to predict sow productivity and assist with decision on culling.

The swine industry needs automated and quantifiable indicators of sow reproductive potential, body condition and locomotion that can be benchmarked with key production indices (KPIs). The current assessments for body condition in sows include physical calipers that, when placed on the last rib, measure body width. Some swine analysis software programs are designed for single farm use or for one application (e.g., thermal temperature). Such approaches do not allow for common management platforms nor the merging of data from different farms and require numerous applications and substantial hardware investment. This lack of integration means that farmers who want to implement more than one technology have to maintain each analysis system separately.

Precision livestock farming (PLF) aims to improve both animal welfare and farmer productivity as well as ease the burden on caregivers. A critical technology enabling this is automated monitoring of individual animals. Currently, methods to measure body condition includes a human utilizing a caliper tool or human observation of locomotion. These modes of evaluation are prone to inconsistencies due to human error, transcription and subjectivity.

Thus, existing attempts at PLF do not achieve precise 3D tracking of animals as they move around a farm. Having this capability would open the door to automated collection and analysis of shape and motion-based health metrics for livestock as disclosed herein.

### BRIEF DESCRIPTION OF THE DRAWINGS

FIG. 1 shows exemplary production facility.

FIG. 2 shows exemplary monitoring device.

FIG. 3 shows exemplary process for estimating a health level of an animal.

FIG. 4 shows exemplary process for estimating motion of an animal.

FIG. 5 shows an exemplary process 500 for training a model to identify abnormal motion in an animal.

FIG. 6A shows an example of skeletal locations identified on a sow in a video frame.

FIG. 6B shows an exemplary pose of a sow identified in a video frame.

FIG. 7 shows an example of a monitoring system.

FIG. 8 shows an exemplary monitoring device positioned in a monitoring area

FIG. 9 shows a depth image of an animal, exhibiting topologies of the animal from a top-down view, as it moves from one room to another in a farming facility, in which landmarks of interest have been tagged or marked with identifiers.

### SUMMARY

In one aspect, a method in accordance with the present disclosure involves analyzing animal health. In particular such a method may comprise acquiring video data of at least one subject animal, the video data comprising a number of video frames, from a monitoring device located at a livestock facility. Based on the video data, an animal of interest is detected. At least one of a topology, a shape, or a gait of the animal is determined, wherein the topology or shape is indicative of a body composition of the animal. The method may also determine whether the topology, shape, and/or gait is abnormal using a trained neural network, then output a notification to a computing device associated with at least one of the facility or a buyer, indicating at least one of the following: an indication of the body composition of the animal; an indication of the gait quality of the animal; a productivity prediction for the animal; or a recommended intervention for the animal.

A method according to this disclosure may also include determining a productivity score of an animal from a measurement of the animal, which may be made using at least one of a depth image, a depth video clip, an IR reading, an IR image, and an optical image. In some embodiments the productivity score may be updated or refined based upon historical sets of measurements of the animal at various locations and times within a farming facility.

In another aspect, the present disclosure includes various systems and apparatus for taking health assessments of animals. Such a system may include a camera (which may be a depth camera, an IR camera, an optical camera, or a combination thereof), a processor, and a memory in communication with the processor. Software instructions stored on the memory, when executed, may cause the processor to: acquire data regarding an animal of interest from the camera during a given time period; determine at least one of a body composition indicator or a pose indicator based on the data acquired from the camera; store the body composition indicator or pose indicator in a data record associated with the animal of interest; and provide the body composition indicator or pose indicator to a neural network trained to predict an animal outcome for animals of a similar species to the animal of interest.

### DETAILED DESCRIPTION

Various systems and methods are disclosed herein for overcoming the disadvantages and limitations of existing approaches.

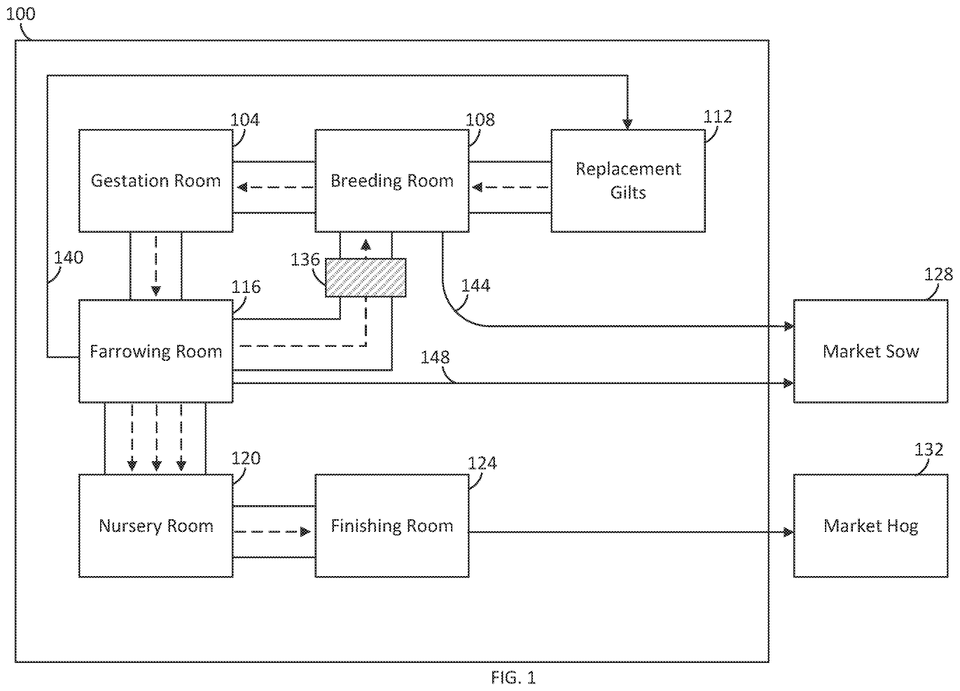

FIG. 1 shows an exemplary commercial livestock production facility 100. In one embodiment, the facility could be a pork production facility 100 that can produce at least one market sow 128 and/or at least one market hog 132. In some embodiments, the production facility 100 can include a gestation room 104, a breeding room 108, a farrowing room 116, a nursery room 120, and/or a finishing room 124. In the breeding room 108, sows from the farrowing room 116 and/or replacement gilts 112 can be bred. In some embodiments, the sows and/or gilts can remain in the breeding room for about twenty-eight to forty days. The sows and/or gilts can leave the breeding room 108 and proceed to the gestation room 104. After leaving the breeding room 108, they gilts can be referred to as sows.

The sows can remain in the gestation room 104 until they are ready to farrow. In some embodiments, the sows can remain in the gestation room 104 for about seventy-five to eighty-seven days. The sows can then proceed to the farrowing room 116. In the farrowing room 116, the sows can give birth to male and/or female pigs. After the sow births the pigs, the male pigs can proceed to the nursery room 120. In some embodiments, at least some of the female pigs can be sent (e.g., at 140) to be used as replacement gilts. In some embodiments, at least some of the female pigs can proceed to the nursery room 120. In some embodiments, the male pigs and/or female pigs can remain in the nursery room 120 for about forty-five days. The male pigs and/or female pigs can then proceed to the finishing room 124. In some embodiments, the male pigs and/or female pigs can remain in the finishing room 124 for about one hundred and sixty-four days. When the male pigs and/or female pigs have grown into the market hogs 132 (e.g., at least two hundred pounds), the market hogs 132 can be sent to slaughter.

Healthy sows can proceed to the breeding room 108. However, unhealthy sows may need to be culled. Certain culled sows can be sent to market (e.g., as the market sows), but some sows may not be healthy enough to be sent to market. Reasons a sow can be culled may include poor body composition and/or poor locomotion (e.g., lameness). For example, a sow exiting the farrowing room 116 may be culled and sent to market at 148 if the sow shows a limp that could affect breeding ability. Additionally, sows in the breeding room 108 that fail to be bred may also be culled and sent to market at step 144.

The production facility 100 can include a monitoring area 136 that can be used with a monitoring device (an example of which will be described below) in order to semi-automatically determine the health of the sows exiting the farrowing room. The monitoring area 136 can be large enough for the monitoring device to capture the gait of a sow and/or enough data to estimate the body composition of the sow.

In other livestock applications, a breeding cycle may involve similar rooms, pens, pastures, or barns through which female animals are moved. For example, beef cattle may be herded through various pens or pastures for feeding, birthing, reproduction, etc. As described below, strategic placement of monitoring devices in accordance with the disclosures herein can provide for a more refined and highly sensitive assessment and recommendation system to aid farmers in both (1) determining when to cull or make other interventions for specific animals; (2) making productivity assessments for given animals; and (2) making herd-level assessments of health attributes and productivity.

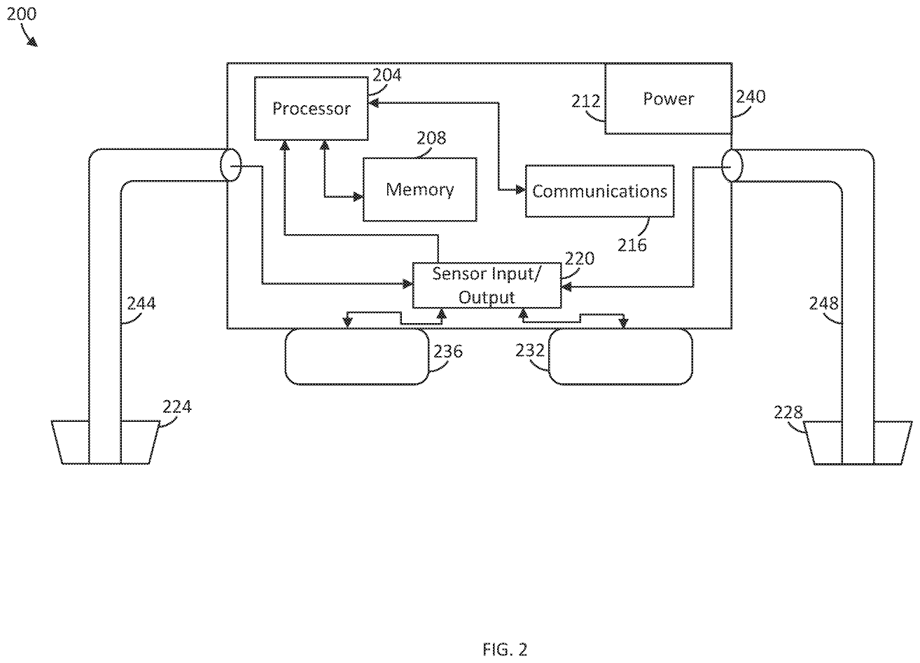

Referring now to FIG. 1 as well as FIG. 2, an exemplary monitoring device 200 is shown. In some embodiments, the monitoring device 200 can include a processor 208, a memory 208, a power source 212, a communication system 216, a sensor input/output module 220, a first infrared camera 224, a second infrared camera 228, and/or at least one supplementary components 232, 236.

The processor 208 can be any suitable hardware processor or combination of processors, such as a central processing unit (“CPU”), a graphics processing unit (“GPU”), etc., which can execute a program, which can include the processes described below. In some embodiments, the communication system 216 can include any suitable hardware, firmware, and/or software for communicating with the other systems, over any suitable communication networks. For example, the communication system 216 can include one or more transceivers, one or more communication chips and/or chip sets, etc. In a more particular example, communication system 216 can include hardware, firmware, and/or software that can be used to establish a coaxial connection, a fiber optic connection, an Ethernet connection, a USB connection, a Wi-Fi connection, a Bluetooth connection, a cellular connection, etc. In some embodiments, the communication system 216 allows the monitoring device 200 to communicate with another monitoring device and/or a computing device (e.g., a server, a desktop computer, a laptop computer, a tablet computer, a smartphone, etc.).

The processor 204 can be coupled to and in communication with the memory 208, the communication module 216, and/or the sensor input/output module 220. In some embodiments, the memory 208 can include any suitable storage device or devices that can be used to store instructions, values, etc., that can be used, for example, by the processor 208 to receive data from the sensor input/output module 220, estimate sow body composition, etc. The memory 208 can include any suitable volatile memory, non-volatile memory, storage, or any suitable combination thereof. For example, the memory 208 can include RAM, ROM, EEPROM, one or more flash drives, one or more hard disks, one or more solid state drives, one or more optical drives, etc.

In some embodiments, the power source 212 can be a battery (e.g., a lithium-ion battery). The battery can allow the monitoring device 200 to be placed in a production facility (e.g., the production facility 100 in FIG. 1) without the need to run additional wiring to the monitoring device 200. In some embodiments, the battery can power the monitoring device 200 for at least two weeks. For biosecurity reasons, certain personnel may not be able to enter a production facility for weeks, and the long-lasting battery can ensure that data is continuously collected between data downloads from the monitoring device 200. In some embodiments, the power source can be a wired power source such as a 12V DC power source, a 120V AC power source. In some embodiments, the power source 212 can include components such as an AC/DC converter and/or a step down transformers to provide DC power to other components of the monitoring device 200 using an AC wall power source.

In some embodiments, at least a portion of the memory 208 can be removable memory such as an SD card and/or a memory stick (e.g. a USB memory stick). In some embodiments, the process 204 can cause the communication system 216 to wirelessly output at least a portion of data generated based on one or more sows (e.g., estimated composition, gait classification, etc.) to an external computing device. For example, the communication system 216 may communicate with the external computing device using Bluetooth protocol. Using either removable memory and/or the communication system to output data to the external communication device can allow the monitoring device 200 to be placed in a production facility without the need to run additional wiring (e.g., an Ethernet cable) to the monitoring device 200. Not requiring the use of physical cables can be especially helpful in large production facilities where a wireless network (e.g., a WiFi network) is infeasible to install due to cost concerns and/or due to the general environmental conditions (e.g., low or high temperatures, moisture, etc.) of the production facility.

The first infrared camera 224 and the second infrared camera 228 can be coupled to the sensor input/output module 220. The first infrared camera 224 and the second infrared camera 228 can be arrange in a complementary position, such as in a stereo formation, which can be used to estimate a distance between a sow and the monitoring device 200. In some embodiments, each of the first infrared camera 224 and the second infrared camera 228 can each be stereoscopic depth cameras. Using multiple depth cameras (which each may be a single sensor/lens or may be stereoscopic) can help ensure that fast moving sows are properly captured by the first infrared camera 224 and/or the second infrared camera 228. In some embodiments, the first infrared camera 224 and/or the second infrared camera 228 can be an Intel RealSense camera (e.g. an Intel RealSense D435 camera). In other embodiments, a single camera could be used, or the first infrared camera 224 and/or the second infrared camera 228 can both be a single-lens camera such as an Azure Kinect DK camera. However, it should be appreciated that the first infrared camera 224 and/or the second infrared camera 228 are not limited to the examples listed above. The first infrared camera 224 and/or the second infrared camera 228 may be any other suitable infrared or depth camera to perform the described steps in this disclosure. It is contemplated that depth image data from these cameras can be obtained in a variety of ways, such as by projecting a field pattern of IR light and measuring the pattern size and dispersion, or by measuring time-of-flight for return detection of IR light, or other means.

In some embodiments, the supplementary components 232, 236 can include an RGB camera, which can be used to provide supplementary data about a sow in addition to any data generated using the first infrared camera 224 and the second infrared camera 228. In some embodiments, the supplementary components 232, 236 can include a light (e.g., an LED lights) in order to provide illumination for an RGB camera. In some embodiments, the supplementary components 232, 236 can include a temperature sensor and/or a humidity sensor in order to generate data about the environment of the production facility where the monitoring device 200 is located. In some embodiments, the supplementary components 232, 236 can include a number of fans that can blow flies and/or other insects away from the first infrared camera 224 and the second infrared camera 228.

In some embodiments, the monitoring device 200 can include a casing including a main portion 240, a first camera arm 244, and a second camera arm 248. The first infrared camera 224 can be coupled to the main portion 240 via the first camera arm 244, and the second infrared camera 228 can be coupled to the main portion 240 via the second camera arm 248. The main portion 240, the first camera arm 244, and the second camera arm 248 can allow the monitoring device to operate in the environment of the production facility, which may be prone to rain or other moisture. Additionally, the portion 240, the first camera arm 244, and the second camera arm 248 can prevent vermin such as mice, insects, etc. from reaching the processor 204, the memory 208, the power source 212, the communication system 216, and/or the sensor input/output module 220.

In some embodiments, the monitoring device 200 can be positioned in order to capture an overhead view of animals such as pigs. In some embodiments, the monitoring device 200 can be positioned in order to capture an overhead view of at least a portion of the monitoring area 136. In some embodiments, the monitoring device can be placed about eight to twelve feet above the ground of the monitoring area 136. In this way, the monitoring device 200 can capture information such as video data of a sow leaving the farrowing room 116.

Additionally, the inventors have discovered that it may be useful in some embodiments to position and direct two cameras 224, 228 so that their field of view only slightly overlaps. This can create a wider or longer field of capture of video data. Therefore, as animals pass in front of, or beneath, the cameras 224, 228, more video frames can be captured showing the animal's gait. The inventors have found that an optimal field of view is obtained by placing the cameras 224, 228 not more than approximately 4 meters away from the animals, and preferably approximately between 2.5 to 1 meters, and more specifically between 1 to 1.5 meters, which would result in a field of view of approximately 2 meters along a hallway for each camera. By orienting the cameras to have a combined 4 meters of field of view along a hallway, corral, or other location through which the animals move, the cameras can capture approximately 1-2 seconds of fast moving animals. Moving the cameras higher, or farther away (e.g., laterally), from the animals would increase the field of view such that the timeframe during which motion tracking takes place would increase. However, depending upon the camera and the conditions within the farming facility, moving the camera farther away from the subject animals could result in a decrease in image quality and/or accuracy of pose prediction. However, for larger animals with more prominent joint features, a farther location may be suitable. A slightly higher positioning may be desirable for beef or dairy cattle, such as 4 meters or greater. For goats raised for milk, their gait is more complex, and so multiple angles of depth video capture may be desirable to detect gait abnormalities. For dairy and beef/brahma breeds of cattle, the more pronounced hip and pin bones in their physiology render capture of their locomotion somewhat easier as compared to pigs, goats, and sheep. Thus, fewer cameras or camera angle captures may be needed. Similarly, the movement of different types of livestock within their typical commercial farming processes lends more or fewer opportunities for assessment and data capture. For example, dairy cattle may move between locations on a farm around 2-3 times per day, whereas pigs may move between rooms of a commercial farm much less during their typical cycles. Likewise, dairy and beef cattle tend to have RFID identification more prevalently in the industry, whereas this is less common for other livestock. This impacts camera needs for animal identification: for example a frontal/facial camera location for obtaining animal identification is less useful when an RFID tag is present.

In post-processing the device can be programmed to combine frames from the two cameras into a timeseries (e.g., some frames of the first camera 224, followed by chronologically subsequent frames of the second camera 228 depending on speed of movement of the animal across the field of view), concatenate the frames from both camera to create one set of wider video frames, or remove overlapping/duplicated content from the two cameras. In alternative embodiments, cameras may be located in two or more separate housings, which are positioned relative to one another to provide additional information. For example, in one embodiment, a one or two-camera monitor may be positioned directly above a hallway of a barn through which sows move (e.g., from room to room) and additional monitors may be positioned to capture video from an orthogonal or profile view. In another embodiment, cameras may be spaced apart and placed in a bar ceiling, but angled at +5 degrees and −5 degrees offsets from a straight downward direction, or +/−10 degree offsets, or +/−20 degree offsets, or +/−30 degree offsets, or +/−45 degree offsets, so that they each capture slightly more profile of the animals passing beneath (rather than merely a direct, top-down view). The output of those cameras could be combined in a “panoramic” or concatenated manner to create one seamless set of video data.

In yet further embodiments, color cameras, UV light cameras, pure infrared (e.g., non-stereo and/or non-depth IR), and other sensors could be included in monitoring device 200. The output of these cameras could be combined with detected gait and body composition data to aid in the discriminatory power of an associated neural network. For example, infrared cameras could be used to monitor individual animals' body temperatures as a measure of animal health or reproduction cycles. Color/visible and UV camera output could be used to detect infections or injuries such as lesions, dermatitis, wounds, and other injuries.

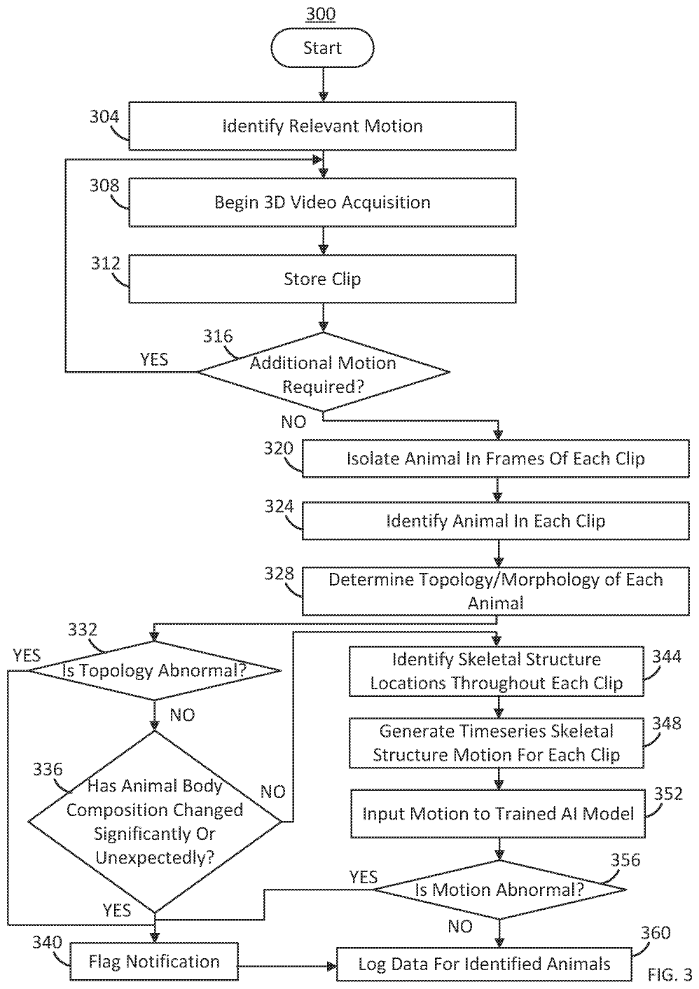

Referring to FIG. 2 as well as FIG. 3, an exemplary process 300 for estimating a health level of an animal is shown. The process 300 can be implemented as computer readable instructions on one or more memories or other non-transitory computer readable media, and executed by one or more processors in communication with the one or more memories or other media. In some embodiments, the process 300 can be implemented as computer readable instructions on the memory 208 and executed by the processor 204. The process can be performed by a processor of a monitoring device according to the disclosure herein, or may be performed via an off-site server (e.g., a cloud computing, or virtual server).

At 304, the process 300 can identify a relevant motion for an animal. For example, a monitoring device may detect animal motion within the device's field of view. In some embodiments, the animal can be a sow. In some embodiments, the motion can be an approximately straightforward walking motion, for example movement down a hallway from one room or pen to another as part of the normal animal movement cycles of a farm. For sows, this may be movement from a gestation room to a farrowing room, or movement from a farrowing room to a weaning room. For cattle, this may be movement from a pasture or feeding area to a barn.

At 308, the process 300 can begin acquiring video data upon detecting animal motion, such as acquiring three dimensional (3D) video data. In some embodiments, the video data can be a stereoscopic infrared video clip a non-stereoscopic infrared video clip, or other series of image frames of a depth sensor. For example, cameras that provide depth data such as Kinect or Intel RealSense, or other cameras that generate depth data from a pattern of projected IR or near-IR, or other light, or LIDAR detectors could be used. In some embodiments, the video clip can be captured using the first infrared camera 224 and/or the second infrared camera 228 of a monitoring device such as monitoring device 200. In some embodiments, the video clip can include a view of the animal. In some embodiments, the view can be an overhead view, an overhead view plus profile view, or a combination of offset angled views (e.g., to capture a slight profile from each side of the animal). The inventors have found that in some instances it may be preferable to obtain direct overhead views, or “down” views, of sows in order to more accurately and efficiently assess certain features and conditions such as body composition, prolapse, and lameness.

At 312, the process 300 can store the video clip acquired at 308. The duration of the video clip may be predetermined (e.g., 5 s, 10 s, or another duration) or may simple continue until motion is no longer detected in the field of view. In some embodiments, the process 300 can cause the video clip to be stored in the memory 208.

At 316, the process 300 can determine if additional motion is required. In some embodiments, the process 300 can determine if enough data has been acquired in order to make an assessment of the animal. In some embodiments, the process 300 can determine if the animal has moved a predetermined threshold distance in the video clip(s) acquired at 308. For example, the process 300 may require that the animal move at least fifteen feet in a direction (e.g., the y-axis direction) before no more movement is required. If the process 300 determines that additional movement is required (i.e., “YES” at 316), the process 300 can proceed to 308. If the process 300 determines that additional movement is not required (i.e., “NO” at 316), the process 300 can proceed to 320. In other embodiments, a more precise positioning of a camera can remove a need to have this step, and all frames of movement of an animal within the field of view can be utilized.

At 320, the process 300 can isolate the animal in each video clip acquired at 308. In some embodiments, the process 300 can isolate the animal using a segmentation technique. For example, the process 300 can provide the video clip(s) to a trained segmentation neural network and receive a number of segmentations indicative of the location of the animal in each frame of the video clip(s) from the neural network. In some embodiments, the process 300 can isolate multiple animals in each video clip or the same animal in multiple frames, and subsequently perform the same analysis on each.

At 324, the process 300 can identify the animal in each video clip acquired at 308. In some embodiments, the process 300 can access a database of known animals (e.g., a database of animals in a production facility) and determine a closest match to the animal isolated at 320. For some farms, pigs transition from room to room at least 3 times during each parity, with 2.2 parities per year on average and up to 6 parities per sow productive lifetime. This offers the opportunity to capture information in a farm system up to 14 times, just using a monitoring device that captures images during pig transitions. Similar transitions occur for other livestock as well, also offering multiple chances to observe animal movements. In embodiments in which multiple cameras are used, a frontal camera could be used to record animal facial features (such as coloring, snout shape, wrinkles, eye size and positioning, and the like) to identify animals using facial recognition and computer vision techniques. In some embodiments, the animals can be pre-marked with a unique identifier such as a code, a number, a pattern, etc. using a marking device as a wax crayon, and the process 300 can identify the animal based on the unique identifier. Wax crayon can be advantageous because it less prone to ingesting by pigs than other identifiers such as tags or physical motion capture markers, and does not interfere with infrared depth cameras. The process 300 can analyze each animal identified at 324 as described at 328-360.

However, in alternative implementations, it may not be necessary, desirable, or feasible to make individual animal identifications. For example, some consortiums or groups of farms may merely want to understand overall herd health and productivity. For example, it may be helpful to understand the percentage of sows in a herd that have optimal, good, or poor body composition—thus it would not be necessary to individually identify each animal as it passes a monitoring device 200. This can also help farmers make more macro-level decisions about feeding, recovery, and other factors for their sow herd.

At 328, the process 300 can determine a topology and/or a morphology of the animal. In some embodiments, the process 300 can provide at least one video frame included in the video clip(s) acquired at 308 to a neural network model trained to estimate if the topology of the animal is abnormal or not. In some embodiments, the process 300 can provide a video frame of the animal (e.g., a depth image of the animal) to a neural network trained to output a score indicative of the body composition of the animal. For example, the process 300 could select a frame of the video clip in which the entire animal is in frame and facing in a uniform (e.g., moving and facing forward) direction. This could be accomplished by, for example, utilizing a computer vision edge detection, color segmenting, or IR “depth” segmenting process (e.g., the floor would always be at a constant distance from the cameras, so the comparative height of an animal could be detected). Once it is determined an entire animal is within frame, a general shape or outline of the animal can be assessed to determine whether the frame shows the animal in a forward-facing posture or otherwise in a position suitable for body composition and gait assessment (e.g., the animal is not lying down, stumbling, or running into another animal). If the animal is not in frame, or is not facing in a suitable direction, then the next frame of the video clip can be considered.

The process 300 can then provide the selected frame to an application that makes an assessment of body composition. In one embodiment, a neural network that has been trained to assess body composition of an animal may be used. The neural network could be a trained network developed through a supervised learning process to detect suboptimal body composition or other indication of a classification of the animal. Or, the neural network could be a single network that simultaneously detects both gait/lameness abnormalities as well as body composition abnormalities. Once the process 300 has received a frame or video data of an animal, it can provide either a score (e.g., how close to an optimal body composition) or a categorization of body composition (e.g., normal/abnormal, or optimal/acceptable/poor, etc.). For example, in some embodiments, the score may be an estimated body fat percentage of the animal. In some embodiments, the estimated body fat percentage can be an estimated back fat thickness. In such embodiment, the trained model may focus (through the supervised learning process) on specific physiological attributes or locations on the animal's body that indicate back fat thickness or other signs of poor body composition. Loss of optimal body condition can be thought of as a combination of loss of muscle and backfat. Currently there are manual tools that measure the level of body condition for pigs, such as an ultrasound system (although these systems have been shown to have a high margin of human error and thus did not have a strong association with reproductive performance, likely because it measured changes in fat layer and excluded muscle loss) and use of a caliper (the caliper has shown more promise because it measures the angularity over the point of the spine between the transverse and lateral process of the spine, although this measurement is time consuming and requires manual intervention for each animal). Therefore, it would be desirable to automate the process of determining backfat as an indicator of body composition, and doing so would have the benefit of uniformity of measurement with farms that continue to use manual caliper methods. In one embodiment, caliper measurements for each animal may be included in a training data set to allow a neural network to learn to associate optimal backfat measurements with the depth and point cloud data over the entire body of the animal that is provided with depth video capture. In another embodiment, a neural net may be trained to capture body composition data more generally from outcome data for each animal.

As another example, in some embodiments, the score may indicate a level of fitness of the animal. The level of fitness may be categorical (e.g., fit or not fit) and/or may be selected from a continuous range of values (e.g., a number ranging from zero to one, inclusive, with zero representing “not fit”, and one representing “fit”). In some embodiments, the process 300 can determine the topology and/or morphology of multiple animals at 328.

At 332, the process 300 can determine if the topology is abnormal. In some embodiments, the process 300 can determine the topology is abnormal if the score received from the neural network is below a predetermined threshold. For example, in some embodiments, the process 300 can determine if an estimated body fat is below a predetermined threshold. As another example, in some embodiments, the process 300 can determine if the estimated back fat thickness is below a predetermined threshold. If the body fat and/or back fat thickness is below a certain amount, the sow may not be fit for breeding because there is not enough fat to sustain the sow during gestation. In some embodiments, the process 300 can determine the topology is abnormal if the score received from the neural network is above a predetermined threshold. For example, in some embodiments, the process 300 can determine the topology is abnormal if the estimated body fat is above a predetermined threshold. As another example, in some embodiments, the process 300 can determine if the estimated back fat thickness is above a predetermined threshold. If the body fat and/or back fat thickness is above a certain amount, the sow may be overweight and at risk of crushing piglets. In some embodiments, the process 300 can determined the topology is abnormal if the score is a discrete value indicating abnormal body composition (e.g., “not fit”). If the score does not meet any of the above qualifiers, the process 300 can determine that the topology is not abnormal. If the process 300 determines that the topology is abnormal (i.e., “YES” at 332), the process 300 can proceed to 336. If the process 300 determines that the topology is not abnormal (i.e., “NO” at 332), the process 300 can proceed to 340.

At 336, the process 300 can determine if the animal body composition has changed significantly and/or unexpectedly. In some embodiments, the process 300 can compare the score to previous scores generated for the animal and determine if the score (e.g., the most recent score) significantly deviates from the previous scores. For example, in some embodiments, if the most recent score is more than two standard deviations away from the average of the previous scores, the process 300 can determine that the animal body composition has changed significantly. As another example, in some embodiments, if the most recent score is more than a predetermined amount (e.g., ten percent) different than the most recent of the previous scores, the process 300 can determine that the animal body composition has changed unexpectedly. If the process 300 determines that the animal body composition has changed significantly and/or unexpectedly (i.e., “YES” at 336), the process 300 can proceed to 340. If the process 300 determines that the animal body composition has not changed significantly and/or unexpectedly (i.e., “NO” at 336), the process 300 can proceed to 344.

At 344, the process 300 can identify a set of structural locations of an animal's body throughout each frame of a video clip. In some embodiments, the process 300 can provide each video frame included in the video clips to a trained model, such as a neural network, which can accurately identify skeletal structure locations of the animal in a given video frame. In another embodiment, a user can manually tag one or more structural locations of an animal's body in a first frame of a video clip (e.g., marking a front shoulder of an animal using a touch screen or cursor), and a neural network can extrapolate other needed structural locations (such as the other front shoulder, hind shoulders, tails, ears, etc.) using various computer vision techniques and trained neural networks as described below. The process 300 can receive, for each video clip, a number of skeletal structure locations from the trained model. In some embodiments, the skeletal structures locations can include a head location, a neck location, a left shoulder location, a right shoulder location, a last rib location, a left thigh location, a right thigh location, and/or an end/tail location.

As an additional assessment, the process 300 could determine a prolapse condition of a sow based upon the determined structural data. For example a measurement could be made from an animal's end/tail to the last rib location, based upon the skeletal structure markings. The inventors have found that this equates to a reliable assessment of prolapse based on IR depth video clips of moving animals. If the distance from the last rib location to the end/tail is greater than a predetermined percentage of overall animal size (e.g., greater than a given percentage of animal length from end of snout to tail base, or from front shoulders to end of animal, or the percentage of animal length from hind shoulders to end of animal/tail base represents more than a predetermined percentage of total animal length, etc.), then the process 300 can flag the animal as potentially having a prolapse condition.

At 348, the process 300 can generate a timeseries of skeletal motion structure for each video clip. In some embodiments, the process 300 can generate a timeseries including coordinate locations for each of the skeletal structures locations for at a number of discrete time points, each time point being associated with a video frame included in a video clip. In some embodiments, the process 300 can generate a single timeseries for every video clip acquired at 308.

At 352, the process 300 can input the timeseries to a trained model. In some embodiments, the trained model can be a trained convolutional neural network. In alternative embodiments, the inventers have discovered that it may be advantageous to utilize an LSTM model to detect abnormalities in animal movement, or otherwise provide an indication of an animal's classification as “optimal,” “suboptimal,” or “gait indicative of likely problem” or “gait indicative of positive health outcome,” etc. In this embodiment, both IR image data as well as skeletal-labeled depth data are provided to the trained model. In this way, the model is trained on both an overall IR “image” of the animal moving, as well as depth data showing timeseries skeletal motion. The model can thus simultaneously provide predictions or scores of body composition as well as gait abnormalities. The trained model can output a score or a classification indication indicative of whether or not the motion exhibited by the animal is abnormal or not (a classification) or can provide merely percentage likelihoods or similar indications that an animal may exhibit a certain characteristic in the future (e.g., poor productivity, poor growth, health issue, etc.). In some embodiments, the score or the classification indication can be a categorical level of abnormality (e.g., abnormal or not abnormal) and/or may be selected from a continuous range of values (e.g., a number ranging from zero to one, inclusive, with zero representing “not abnormal”, and one representing “abnormal.”

At 356, the process 300 can determine if the motion exhibited by the animal is abnormal. In some embodiments, the process 300 can determine that the motion is abnormal if the score output at 352 falls into an abnormal category (e.g “abnormal”). In some embodiments, the process 300 can determine that the motion is abnormal if the score output at 352 is above a predetermined threshold (e.g 0.6). If the process 300 determines that the motion is abnormal (i.e., “YES” at 356), the process 300 can proceed to 340. If the process 300 determines that the motion is not abnormal (i.e., “NO” at 356), the process 300 can proceed to 360.

For animals like sows that are kept largely for reproductive productivity, a fitness level could be dynamically set by a monitoring system and updated for a given herd. For example, weighted thresholds for the topology, gait, body composition, and other characteristics of the top 50% or top 40% or top 30% or top 20% or top 10% of sows by piglet productivity could be determined, and those thresholds could be utilized to determine whether a given animal is optimal or suboptimal in condition. Alternatively, characteristics of the bottom 10%, 20%, 30%, etc. of sows by piglet productivity could be determined and used to determine whether a given sow has a suboptimal or poor condition. On a characteristic by characteristic basis, such as for gait, a monitoring system using a neural network as described herein could be trained to assess gait characteristics that are common to low producing animals, and either output a confidence or similarity score (e.g., “this animal's gait is 90% similar to animals that turn out to have a health issue or low productivity, and 40% similar to animals that have a good gait or that turn out to have good health and productivity”) or simply categorize the animal as “abnormal gait” or “normal gait”. Similarly, for animals that are kept for body mass or other types of productivity (e.g., egg laying or wool growth), a neural network can be trained to determine whether an given animal's characteristics are optimal or suboptimal, abnormal or normal, based upon final productivity measurements or upon eventual illness diagnoses.

In yet other embodiments, a neural network could be trained in an unsupervised manner, or with limited supervision (e.g., by emphasizing or weighting data records that exhibit good animal productivity, body composition, relative health (no illnesses), etc.), such that it would learn to categorize and identify classification indicators of animals.

At 360, the process 300 can log data for identified animals. In some embodiments, for each animal identified at 324, the process can log estimated body composition scores, animal skeletal structure motion, and/or any other data generated at 328-56. The data can be logged to a memory (e.g., the memory 208).

At 340, the process 300 can output a flag notification. In some embodiments, the flag notification can be output to a computing device such as a smartphone. If the process 300 proceeded to 340 from 332, the process 300 can output a flag notification indicating that the topology of the animal is abnormal and/or that the animal should be culled and sent to market. If the process 300 proceeded to 340 from 336, the process 300 can output a flag notification indicating that the body composition of the animal has changed significantly and/or unexpectedly, that the animal should be examined, and/or that the animal should be culled and sent to market. If the process 300 proceeded to 340 from 356, the process 300 can output a flag notification indicating that the motion of the animal is abnormal and/or that the animal should be culled without being sent to market.

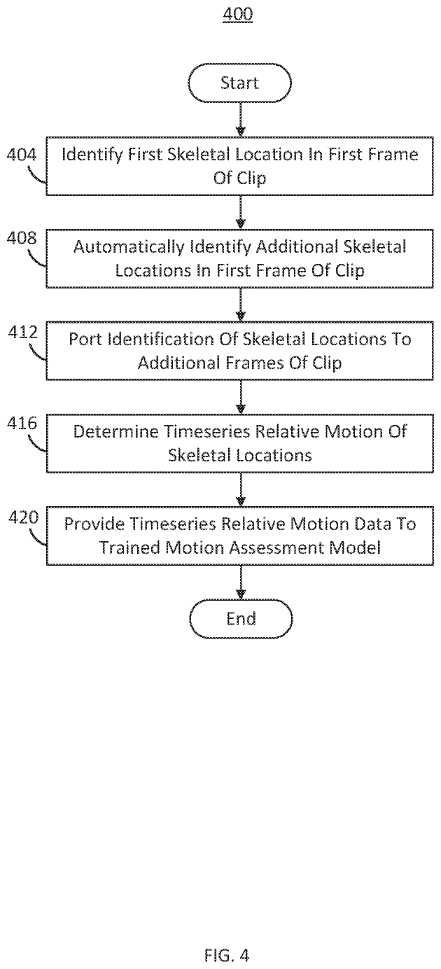

Referring to FIG. 2 as well as FIG. 4, an exemplary process 400 for estimating motion of an animal is shown. The process 400 can be implemented as computer readable instructions on one or more memories or other non-transitory computer readable media, and executed by one or more processors in communication with the one or more memories or other media. In some embodiments, the process 400 can be implemented as computer readable instructions on the memory 208 and executed by the processor 204. The process below could be utilized to determine the timeseries skeletal structure/frame data that is provided to the neural network in process 300 for determining attributes of an animal such as gait abnormality or prolapse.

At 404, the process 400 can identify a first skeletal location in a first frame of a video clip. The first frame can include an overhead view of an animal such as a sow. In some embodiments, the process 400 can identify a marking on the animal. In some embodiments, the marking can be a symbol such as a dot. The marking can be pre-applied to the animal using a wax crayon, which does not interfere with the ability of infrared depth cameras to generate 3D video. The animal may have been marked at a number of locations such as a head location, a neck location, a left shoulder location, a right shoulder location, a last rib location, a left thigh location, a right thigh location, and/or a tail location. The process 400 may identify a specific location (e.g., a head location) as the first skeletal location. In some embodiments, the process 400 can provide the first frame to a trained model (e.g., a neural network) and receive an indication of a coordinate location of the first skeletal location.

At 408, the process 400 can automatically identify additional skeletal locations in the first frame of the clip. Based on the first skeletal location, the process 400 can determine the additional skeletal locations in the first frame of the clip. In some embodiments, the process 400 can provide the first frame of the clip and the location of the first skeletal location to a trained model such as a neural network and receive the additional skeletal locations from the trained model.

At 412, the process 400 can port the identified skeletal location in the first frame to additional frames of the video clip. In some embodiments, the process 400 can utilize pairwise optical flow to propagate the skeletal locations in the first frame forward and backward through a sequence using Deepflow. Deepflow has high accuracy with large displacements which can occur when pigs run. However, if only optical flow were used to propagate labels, mark locations may drift and error may accumulate. The process 400 can use physical markings (e.g., wax crayon markings) for location and the optical flow only for propagating marker identification.

At 416, the process 400 can determine a timeseries of relative motion of the skeletal locations. The timeseries can include the coordinate locations for each of the skeletal structures locations for at a number of discrete time points, each time point being associated with a video frame included in a video clip.

At 420, the process 400 can provide the timeseries of relative motion of the skeletal locations to a trained motion assessment model. The trained motion assessment model can output a score indicative of the quality of motion of the animal (e.g., “abnormal,” “not abnormal”) based on the timeseries.

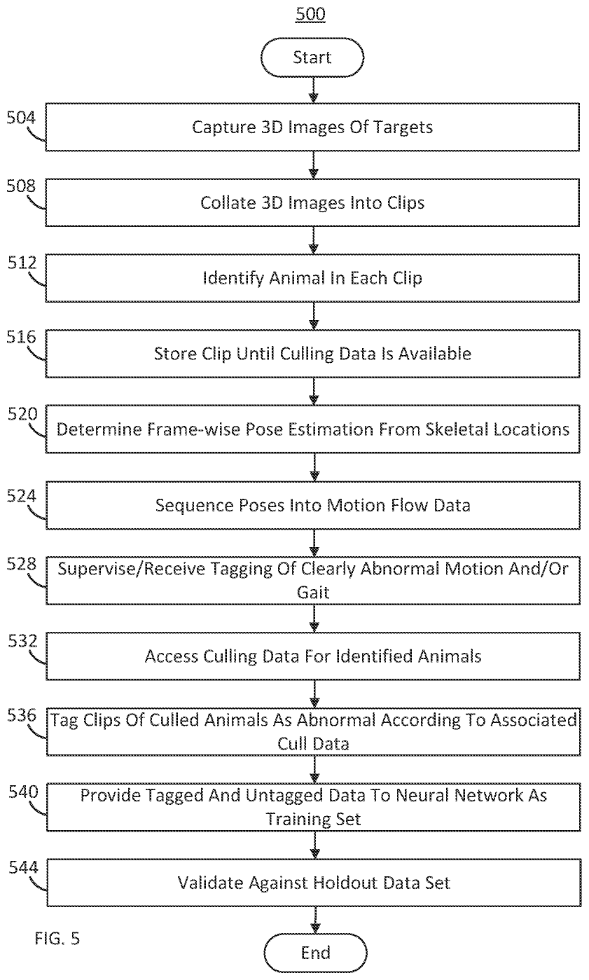

FIG. 5 shows an exemplary process 500 for training a model to identify abnormal body composition, abnormal gait/motion, or other abnormal attributes in an animal. The process 500 can be implemented as computer readable instructions on one or more memories or other non-transitory computer readable media, and executed by one or more processors in communication with the one or more memories or other media. In some embodiments, the process 500 can be implemented as computer readable instructions on the memory 208 and executed by the processor 204.

At the start of training process 500, a dataset comprising IR and depth video data (which may be generated from an IR depth sensor) of livestock of interest is obtained at step 504. In one embodiment, the dataset may be obtained from an association of one or more livestock barns of one or more farms, or from an entire consortium or co-op. For example at each barn, one or more devices according to the disclosure herein can be used to capture 3D image and/or video data of target animals. For example, infrared and depth image and/or video data can be obtained. In some embodiments, the targets can be animals such as pigs. At 508, the process 500 can collate and segment the data into discrete video clips. For example, in one embodiment, video capture may be continuous, but acquired frames of data are only stored (e.g., in increments of a few seconds, 10 s, 30 s, etc.) if motion is detected. In another embodiment, video/data capture may be motion sensitive or turned on manually when movement of livestock will be permitted. The associated clips can then be processed on a timeseries basis, to determine which frames of a given video clip likely contain an animal.

At 512, the process 500 can identify an animal in each video clip. In some embodiments, the process 500 can identify whether an animal of interest exists in a video clip by performing a background removal process, then applying a trained machine learning algorithm (e.g., a trained convolutional neural network) to quickly identify whether the object in the image (after background remove) is, e.g., a pig or not. In other embodiments, a priori knowledge of which animals will be moving in a given space within a bar can remove any need to perform an analysis of the type of animal in an image. In other embodiments, the specific identity of the individual animal in a clip can be assessed using a computer vision process to determine the presence of a unique identifier (e.g., a serial number or barcode) marked on the animal (e.g., using a wax crayon).

At 516, the process 500 can store the video clips until outcome or diagnosis data is available for the animals in the clips. In one example, individual animals are identified during the algorithm training process 500, and video clips of those specific animals are associated with various health, productivity, or outcome data for that specific animal. In one example, early culling of a sow may be used as a metric for that animal's outcome. In other words, if a sow is culled and sent to market before an expected age or expected number of reproduction cycles, it can roughly be assumed that there was a problem identified by the farmers (which could have to do with physiological signs of distress, lameness, being undersized or not eating, having low or no piglet productivity, needing a lengthy recovery cycles, or another indication of severe or non-optimal condition). Sows with this outcome could be tagged as “abnormal.” Sows who are sent to market at an expected age or number of cycles would be tagged as “normal.” The inventors have determined that this sort of outcome data, when associated with video data for individual animals from a large dataset, can be used to train an algorithm to accurately identify animals with health issues earlier, from more subtle indications, faster, more efficiently, and more accurately than an average human could identify. In other embodiments, more granular information about an animal's health or productivity can be used to train an algorithm. For example, data for an individual sow that could be gathered and associated with that sow's video data are: number of birthing cycles, average size of piglet litter, total number of piglets, weight at market time, time between litters, involvement in aggressive behaviors or fighting, and body composition measurements such as back fat thickness.

At 520, the process 500 can determine a number of frame-wise pose estimations from skeletal location of the animal. In some embodiments, the process 500 can implement at least a portion of steps 404-412 at 520. Alternatively, a user working to train the model could provide identifications of structural locations in frames of video clips by manual marking. Or, a blended approach could be taken in which an algorithm predicts structural locations in each frame and a user simply confirms or adjusts the predicted skeletal locations via a user interface.

At 524, the process 500 can sequence the pose estimations into motion flow data. In some embodiments, the process 500 can implement at least a portion of 416 at 524.

At 528, the process 500 can label motion flow data as either normal (control) or abnormal (case). This can be done in a variety of ways including manual or supervised learning (e.g., users tagging the actual video clips as showing abnormal gait), and/or using subsequent outcome data. In the latter case, the model would be provided with outcomes of each animal that is represented in the motion flow data. The outcome data provides an indication of whether the animal remained healthy, at proper weight and body composition, and was sent to market at the normal or expected time—in other words a healthy, normal animal with a typical outcome. For other animals, the outcome data might indicate the animal wound up having a suboptimal weight, became sick, exhibited physiological distress or injury, and was culled early or some other atypical intervention was taken as a result. These outcomes may be recorded into a database by users at the farming facility, based upon their own current criterial for culling or other intervention. In this manner, the machine learning model is “trained” to recognize suboptimal animal body composition and gait characteristics associated with atypical outcomes.

At 532, the process 500 can access culling data for the identified animals. The culling data can be real-world information indicating whether the animal was culled after the video clip was captured. In other embodiments, more specific outcomes can be associated with the animal motion data. For example, lower weight, longer recovery periods, and other suboptimal outcomes can be associated with animal data beyond simply early culling.

At 536, the process 500 can tag video clips of culled animals as abnormal according to several inputs. In the inventors' experience, it was useful to employ a combination of manual tagging of video clips with final outcome data in order to train a neural network model, such as an LSTM model. In this approach, an individual (such as the farmer, a livestock veterinarian, or other knowledgeable individual) would tag clips of animals in which an abnormality could clearly be identified. For example, the individual could tag a video clip as exhibiting lameness, other abnormal gait, overweight, underweight, prolapse, and/or other indicators of a poor health condition. In addition to this tagged information, final outcome data of animals in the individual video clips could be used as a proxy for an abnormality. The final outcome data may correlate to all animals in the training data set, or to only some animals, and may overlap partially or wholly with the manual tagging. Doing so provides several benefits, including confirmation that abnormalities exhibited by an animal did in fact cause a suboptimal outcome for that animal, and providing a faster and more efficient way to obtain larger training datasets without having to resource knowledgeable individuals to manually tag video clips. In one embodiment, video clips associated with animals that ultimately were culled early (or for which other interventions were taken) could be prioritized and provided to a knowledgeable user for review for manual tagging of more specific attributes such as which leg exhibited lameness, etc. For video clips that are associated with (1) a user's manual tagging of an abnormality plus (2) an early cull outcome, a higher weighting could be given in the training dataset. In the inventors' experiments, the trained model is able to accurately identify and predict animals that will ultimately need early culling or other intervention earlier and using more subtle cues than current manual or electronic methods.

At 540, the process 500 can provide tagged and untagged (i.e. normal/healthy animal) data to a neural network as a training set. The neural network can be trained to identify abnormal motion and/or gait based on the tagged data (which can indicate abnormality) and the untagged data (which can indicate lack of abnormality).

At 544, the process 500 can validate the neural network against a holdout data set.

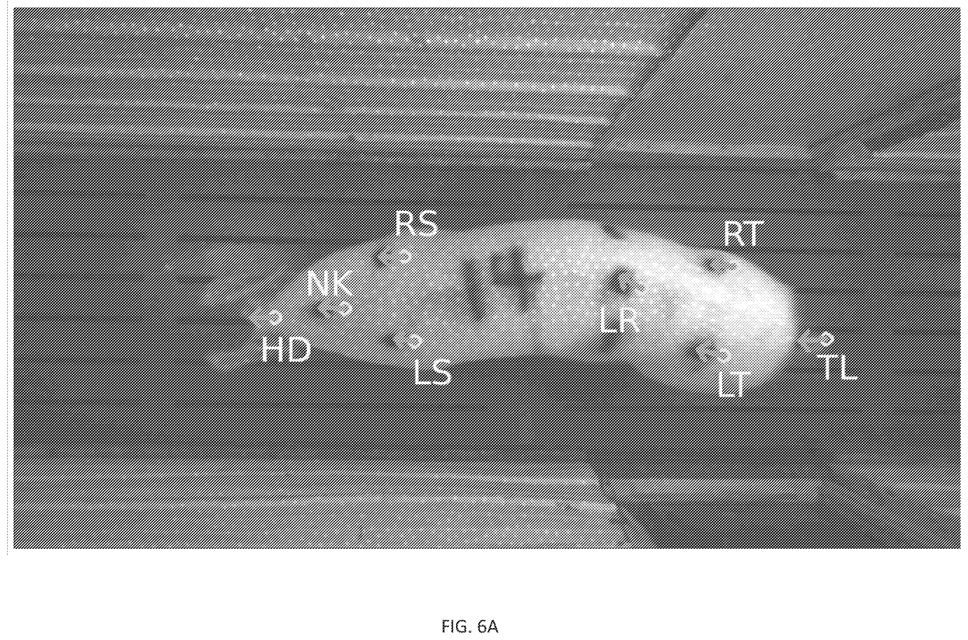

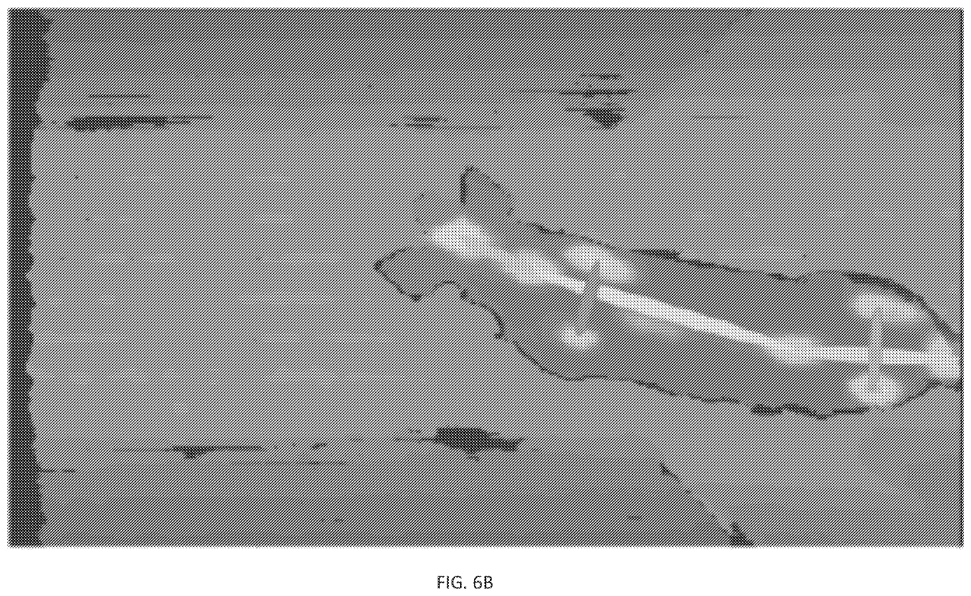

FIG. 6A shows an example of skeletal locations identified on a sow in a video frame. The skeletal locations include a head location, a neck location, a left shoulder location, a right shoulder location, a last rib location, a left thigh location, a right thigh location, and/or a tail location.

FIG. 6B shows an exemplary pose of a sow identified in a video frame. As described above, the wireframe skeletal/structural data obtained from tagging an animal per the process 400 described above can be utilized to develop timeseries motion data representative of an animal's gait while moving from room to room or pen to pen within a livestock farming facility.

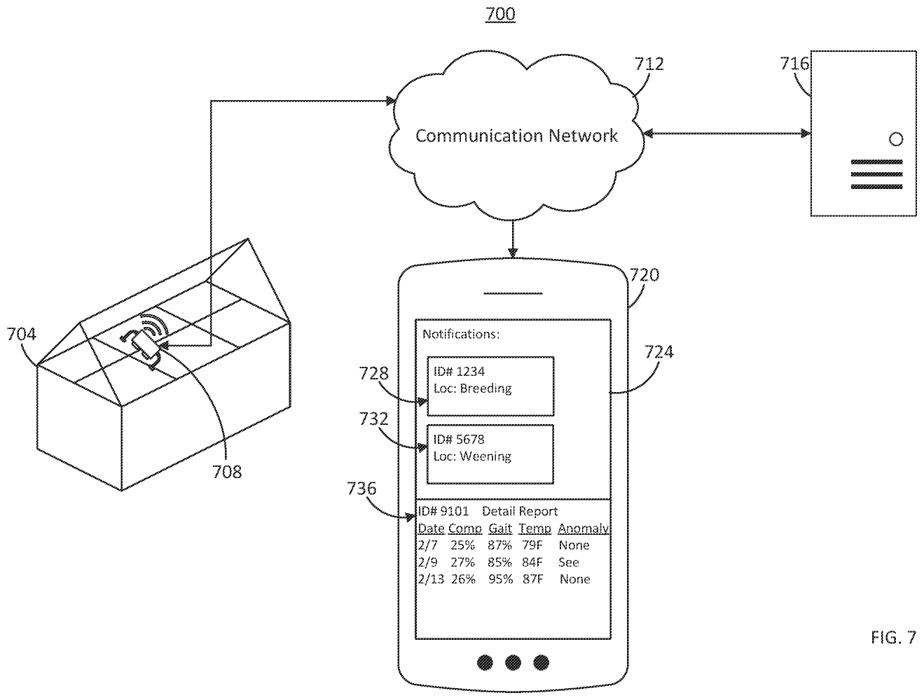

FIG. 7 shows an example of a monitoring system 700. The system 700 can include one or a network of monitoring devices 708 positioned in one or a network of production facilities 704, a server 716, and a computing device 720 in communication over a communication network 712. In some embodiments, the communication network can be a wired network (e.g., an Ethernet network) and/or a wireless network (e.g., Bluetooth, WiFi, etc.). The monitoring device 708 can output data including raw data and/or estimations as well as notifications to the server 716 and/or the computing device 720. The monitoring device 708 can implement at least a portion of the process 300. The server 716 can store at least a portion of data output from the monitoring device 708.

### Example: Sow Monitoring and Gilt Assessment

One advantageous implementation of the present disclosure is found in a system configured to monitor gilts and sows in a commercial farming operation. By accumulating multiple categories of physiological and performance characteristics of an animal throughout its lifecycle from gilt to sow, a model can be trained to provide real time assessments of animal health as well as predictions of future productivity.

The inventors have determined that it may be advantageous in some circumstances to utilize monitoring devices 708 located in multiple barns of a given farm, or even across multiple farms to monitor gilts and sows. As these devices 708 record data regarding the animals (which may include, for example, body temperature, size, body composition, litter size, number of farrowing cycles, and gait/motion data) the monitoring devices 708 can provide real time predictions/assessments of animal health and predictors of animal productivity. In one embodiment, an assessment is made of the size, weight, shape, topology, and movement of a gilt as it moves from (or is ready to move from) a growing zone. For example, a size of the animal may be determined from a depth camera, IR, camera or other similar sensor, by for example determining the size of the animal's profile, or calculating a volume from the output of the depth sensor. A weight of the animal could be determined from a scale or other weight sensor, or could be calculated from output of an optical or depth camera. In one embodiment, a data set of animal images can be correlated with measured animal weights. A regression or neural net can be trained to accurately estimate weight from the dataset. A shape and topology of the animal can be taken from a depth camera output. And, the animal's movement can be assessed as discussed above. Finally, other statistics concerning the animal's productivity can be entered into the record manually by a user (e.g., via device 720) or can be automatically determined. For example, an optical camera positioned over crates or pens of a farrowing room could be utilized to detect and count the number of piglets per animal, and the numbers could be stored in the animal's record.

At each point in facility 704 at which a monitoring device 708 records information concerning an animal, the animal's identity is determined (e.g., through camera detection of a marker, through use of an RFID tag, or through use of image recognition methods), and the measurements and assessments acquired are then stored in a memory. As that specific animal passes through other regions of the farm throughout its lifecycle, the same measurements and data acquisition are made. With reference to FIG. 1, monitoring devices may be placed at various combinations of the entrance, exit, or inside the gilt room 112, breeding room 108, gestation room 104, and/or farrowing room 116. In some facilities additional rooms may also exist, such as growing or recovery rooms for sows post-farrowing who are not yet ready for breeding. The data records of the animal (including clips of the animal's gait throughout its lifecycle) can be compiled and used to train a predictive neural network. Optionally, a user may flag an animal's record as being non-informative if the animal was injured (e.g., through fighting) or some other unexpected or uncontrollable situation occurred that resulted in lameness or decreased productivity for the animal.

In this manner, the training dataset of gilt/sow lifecycle records can be curated to ensure a higher predictive power is achieved based on the animal's characteristics at a gilt stage. One goal of such a system could be to train a neural network (such as a CNN, RNN or LSTM network) to assess gilt attributes (gait, speed, size, body composition, etc.) and make a predictive assessment of which animals may turn out to be outliers in the sense of likelihood it will be unproductive or unhealthy as it becomes a sow and enters the breeding cycles.

Additionally, a neural network could be trained on animals' records (or partial record, such as body composition and gait) for a given farm, group of farms, or other collaboration to make early assessments of an animal's health and productivity trajectory. For example, after a first farrowing and recovery, an adult sow could be assessed to determine whether further breeding would be productive for that animal.

The devices 708 can also be utilized as sources of additional training data to further refine the trained neural network that makes those predictions/assessments. For example, as animals are culled early or other interventions are taken, farmers at each location can utilize a computing device 720 to associate outcome data with each animal.

The computing device 720 can include a display 724. In some embodiments, the computing device 720 can be a smartphone. The computing device 720 can implement a graphical user interface (GUI) in order to display a number of notifications and/or a detailed report 736 associated with a specific animal. A first notification 728 can be associated with a first animal, and the second notification can be associated with a second animal. Each notification 728, 732 can include animal characteristic information such as an animal identification number, a location of the animal, and/or a status of the animal (e.g., abnormal gait, abnormal topology). The detailed report 736 can include historical information about an animal, such as a date the animal was analyzed, estimated body composition, a score indicative of gait, weather information (e.g., temperature, humidity, etc.) of the day the animal was analyzed, and/or abnormality information.

The computing device 720 can also include a GUI for inputting animal outcome or interventional data. For example, if a farmer determines that a given sow or heifer needs to remain in a recovery pen after farrowing for a longer period of time, the farmer can enter the animal's ID number (e.g., from an ear tag, branding, or wax crayon marking) and select from among a list of outcomes/interventions such as early culling, longer rest time, additional feed, less feed, or the like. This outcome data, when added to a record for the animal that also includes acquired movement and body composition data, can be utilized as additional training data to further refine the neural network model. Similarly, a farmer could enter the number of piglets per litter for each animal and the number of litters. Likewise, a farmer could indicate when the animal is sent to market and final market weight/size.

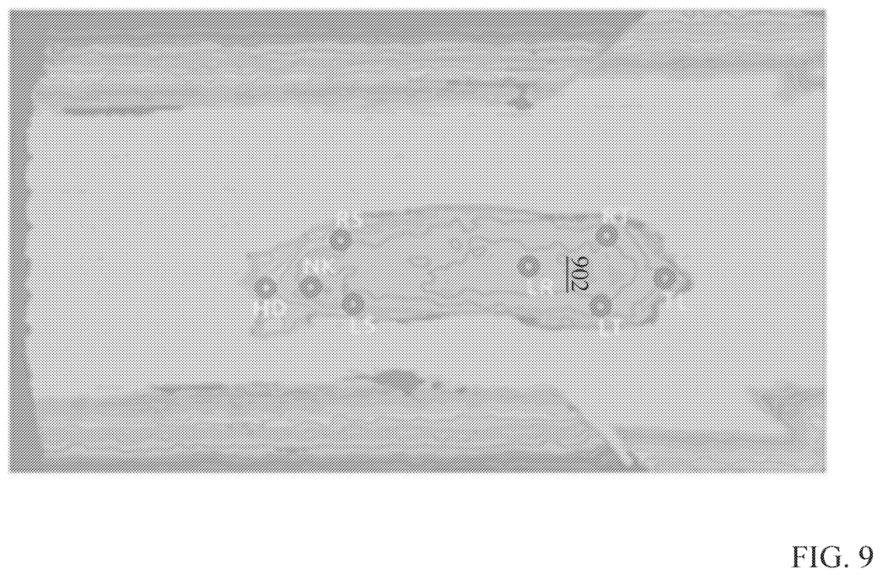

In alternative implementations, one or more additional monitoring devices could be positioned within a barn so that animals are observed by the cameras and/or other sensors at additional points in the farming cycle. For example, a monitoring device could be positioned at an exit of a barn to identify and measure the body composition, size, and health of animals being sent to market for slaughter (both hogs and sows). For market hogs, a measurement of body composition could be made just before the animal is ready to be sent to market. In one embodiment, the inventors have determined that it may be advantageous to make measurements of size (e.g., height, length from snout to tail, height/width at shoulder, or other desirable characteristics), weight, body composition, backfat, and other similar measurements. A backfat measurement could be made by, e.g., making an assessment of the width of the highest point of an animal's back. With reference to FIG. 9, a topological or depth image of an animal is shown. This image could be a single image or a series of frames of a video clip taken of the animal moving. In the case of a single image, a measurement could be made of the width of the highest point or region 902 of the animal's back. For example, this width could be consistently measured at the last rib location LR or between the left and right hind shoulders RT, LT. In the case of a video clip, the frame most likely to be a centered, top-down view could be utilized for the measurement (e.g., as determined by whether the topological depth changes of the animal are roughly symmetrical or mirrored along a center line of the animal's image) at the point of measurement or along the animal's entire back or spine. In other embodiments, an average measurement could be determined from all frames of an image.

Data concerning the size, body composition, weight, backfat, and/or other measurements of a given hog or batch of hogs could then be sent to or matched to potential buyers. For example, a given slaughtering operation may desire hogs of a certain size or weight range to maximize efficiency of their processes, or may be willing to pay a higher price for animals having an optimal body composition (e.g., muscle to fat ratio as estimated from weight, size, and backfat). Batches of hogs from a farming facility 704 could then be automatically determined to meet the desirable buying criteria. The batch's attributes could be stored to a blockchain record (either individual animal attributes of the batch, or averages, medians/quartiles, etc.) to follow the batch and slaughtered and processed pork from the batch.

As another example, a monitoring device could be positioned in a farrowing room or at the exit of a farrowing room to identify the number of piglets per animal (on an individual or herd basis). Alternatively, a monitoring device could be positioned at the exit of other rooms, such as the gestation room 104, breeding room 108, a farrowing room, a nursery room 120, and/or finishing room 124 to capture additional information about an animal. For example, the animal's movement from room to room could be utilized to calculate certain criteria like weened estrus interval. In one example, sows may be moved from a breeding room from a farrowing room. If the sows do not return to estrus within seven days, it can be taken as an indicator of poor reproductive capability and an indicator the animal may need to be culled. Similarly, animals that resist moving to a breeding room from a farrowing room may indicate they are having difficulty with breeding or recovery. As sufficient training data records are obtained in this manner, including from multiple barns/farms, the model can be updated and validated across barns.

In a related embodiment, when sows are culled and it is determined they will go to market, information concerning the sow's current health and projected health can be utilized to determine which sows would be the best candidates for being sent to various slaughter operations. Often, sows are older and larger than market hogs at the time they are ready to be shipped to slaughter. And, the time to slaughter for sows can be much longer than the time to slaughter for hogs—in some instances the time from culling to slaughter for market sows can be as long as two months, whereas the time is more typically a few days for market hogs. Therefore, being able to project the future health of an animal and its likely ability to endure the shipping process can be much more important for sows than hogs. Based on a market sow's size, weight, backfat, gait, and other similar attributes, buyers located in more distant areas (e.g., where shipping times might be significantly increased) may be able to select healthier animals at a slightly higher price. Similarly, given the weight of sows, some shippers may be able to more efficiently load animals on to trucks if sizes and weights are known in advance. Accordingly, in one embodiment, market sows are measured by a measuring device 708 as the enter a market sow room 128. The measurement device 708 may acquire a depth camera video clip, an IR temperature measurement, and measurements of animal size and backfat made from the depth image. Gait abnormalities may be assessed from the video clip as described above. This current data may optionally be associated with historical health information regarding the animal, such as whether it had a history of illness or injury, whether it had difficulties in recovering from farrowing (e.g., a long weened estrus cycle), exhibited consistent low weight, an unwillingness to leave the farrowing room, etc. Additionally, health and productivity predictions for the animal throughout its life may also be included—such as, e.g., the percentage predictions of productive outcomes for the animal made by a neural network based on measurements taken post-farrowing, at gilt stage, or other stages during its life. These scores may be thought of as positive indicators of health or productivity. More objective criteria such as body composition scores could also be included.

As discussed above with respect to market hogs, batches of market sows (or individual market sows) could have associated characteristic data stored in a blockchain record and sent to or matched to potential buyers. This data could also be used by shippers to more intelligently load trucks that may make multiple stops—for example, the sows with the lowest/worst indicators of health (poor gait, poor body weight, poor health history, etc.) could be loaded so that they are unloaded first. Similarly, based on animal size, an appropriate plan for feeding the market sows during transportation could be made.

Beyond simply providing indicators and predictions of current animal health, the system could also provide recommendations to farmers. For example, if an animal is detected as having prolapse or a long weened estrus interval, the system could send a notification to device 720 with the recommendation to cull the animal. In other instances, if an animal is slightly below weight after farrowing, the system could recommend that the animal be given additional time to gain weight before returning to the breeding room. Similarly, animals that exhibit poor traits as gelts could be removed from the breeding pool right away.

In another embodiment, general health and productivity data by herd could be obtained from a given barn or farm, rather than or in addition to individual animal health/productivity. For example, data for a given farm or a given “batch” of animals could be collected indicating statistics regarding body composition, such as average body composition, the distribution of animals above weight, severely above weight, below weight, and/or severely below weight. Or, as another example, statistics regarding back fat thickness could also be determined. This information could be used in several ways. First, the data could be associated with a blockchain record for all meat coming from that batch. Second, the data could be correlated with a profile for a given farm that includes geographic/weather/climate information, as well as animal breed/subspecies type, feeding and exercise practices for the given farm, and similar information about how the animals were raised. This data could then be regressed over time to give comparable farms within a common network recommendations for more efficient feeding and other relationships between farming practices and animal health and productivity.

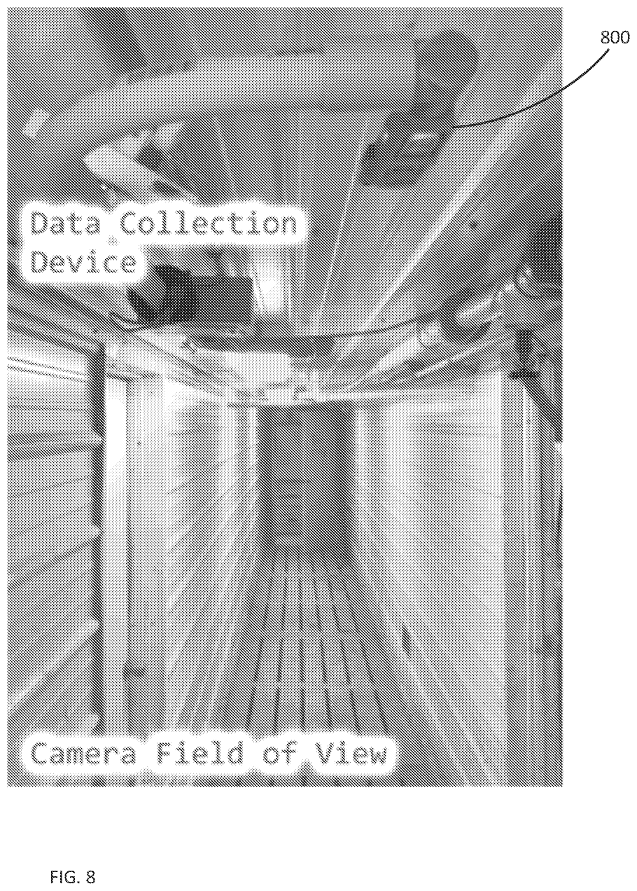

FIG. 8 shows an exemplary monitoring device 800 positioned in a monitoring area. The monitoring device 800 can be positioned about eight to twelve feet above the floor of the monitoring area, and oriented to capture a downward facing view of a portion of the monitoring area. As shown, the monitoring device is positioned over a hallway through which sows move from one room to the next. It should be understood that such a monitoring device could also be placed over gates or entryways between pens, pastures, barns, milking facilities, breeding areas, hatching/laying rooms, or other discrete sections of a livestock farm. And, the monitoring device 800 could comprise one or more units that are positioned at the ceiling at various angles relative to the animals moving along the hallway. For purposes of durability and stability, the inventors have determined it is advisable to position the monitoring device(s) 800 out of the reach of the animals.

Various designs, implementations, and associated examples and evaluations of a system for automatic livestock analysis are described above. However, it is to be understood the present invention has been described in terms of one or more preferred embodiments, and it should be appreciated that many equivalents, alternatives, variations, and modifications, aside from those expressly stated, are possible and within the scope of the invention.

Example 1. A method for analyzing animal health, the method comprising: acquiring a sequence of depth images of at least one subject, from a monitoring device located at a facility; detecting a subject in the sequence of depth images and identifying a class of the subject; determining at least one of a topology of the subject, a gait of the subject, or a body composition of the subject based on the depth images; determining a classification indication for the subject relating to a set of potential classifications based on the class of the subject and at least one of the topology of the animal, the gait of the animal, or the body composition of the animal using a trained neural network; and outputting a notification based on the classification indication to a computing device associated with at least one of the facility or a buyer, the notification indicating at least one of the following: an indication of the body composition of the subject; an indication of the gait quality of the subject; a productivity prediction for the subject; or a recommended intervention for the subject.

Example 2. The method of Example 1, wherein the category of the plurality of categories is determined based on a score between a continuous range of scores.

Example 3. The method of Example 1, wherein the category of the plurality of categories is determined based on previously determined categories on at least one of previous topologies, shapes, gaits, or body compositions.

Example 4. The method of Example 2, wherein the category of the plurality of categories is further determined based on a threshold to compare the at least one of the topology of the animal, the shape of the animal, the gait of the animal, or the body composition of the animal with the threshold.

Example 5. The method of Example 1, wherein the gait of the animal is determined by: identifying a joint in a first frame of the number of video frames with a mark; porting the identified joint in the first frame to a second frame of the number of video frames; determining a time-series relative motion of the joint based on the joint in the first frame and the joint in the second frame; and determining the gait of the animal based on the time-series relative motion.

Example 6. The method of Example 5, wherein the gait of the animal is provided to the neural network trained to identify categories of the gait, and wherein the neural network was trained on a dataset comprising previous animal gait information and the categories in connection of the previous animal gait information.

Example 7. The method of Example 1, further comprising: determining an indicator of the animal's backfat by measuring a region of the animal from the video data.

Example 8. The method of Example 1, further comprising: determining an indicator of the body composition of the animal by determining at least one of a height, shoulder width, estimated weight, and estimated volume of the animal from the video data.

Example 9. A precision livestock farming system comprising: a camera; a processor; and a memory in communication with the processor, having stored thereon a set of instructions which, when executed, cause the processor to: acquire data regarding an animal of interest from the camera during a given time period; determine at least one of a body composition indicator or a pose indicator based on the data acquired from the camera; store the body composition indicator or pose indicator in a data record associated with the animal of interest; and provide the body composition indicator or pose indicator to a neural network trained to predict an animal outcome for animals of a similar species to the animal of interest.

Example 10. The system of Example 9, wherein the camera is a depth camera.

Example 11. The system of Example 10, wherein determining at least one of a body composition indicator or a pose indicator comprises determining landmarks of interest in a depth image of the animal of interest.

Example 12. The system of Example 11, wherein determining landmarks of interest in the depth image further comprises using a landmark detector to identify landmarks of interest in another image of the animal of interest and transposing the landmarks of interest to the depth image.

Example 13. The system of Example 9, wherein the neural network is trained to predict whether the animal of interest will exhibit an abnormal gait based upon a timeseries of depth image frames of a video clip of the animal of interest.

Example 14. The system of Example 9, wherein the processor is further caused to output a notification to the farming facility identifying a health issue for the animal of interest based upon the output of the neural network.

Example 15. The system of Example 9, wherein: the camera is a near-infrared depth camera positioned within farming facility; the processor is further caused to: determine a gait abnormality for a batch of animals from a set of depth video clips of batch of animals acquired by the camera; determine body composition scores of the batch of animals based upon at least one of a height, shape, backfat width, or volume of each animal of the batch of animals; output the gait abnormality and body composition determinations to at least one of a network associated with the farming facility or a network associated with potential buyers of the batch of animals.

What is claimed is:

1. A method for analyzing animal health, the method comprising:
acquiring a sequence of depth images of at least one subject, from a monitoring device located at a facility;
detecting a subject in the sequence of depth images and identifying a class of the subject;
characterized by:
determining at least one of a topology of the subject, a gait of the subject, or a body composition of the subject based on the depth images;
determining a classification indication for the subject relating to a set of potential classifications based on the class of the subject and at least one of the topology of the animal, the gait of the animal, or the body composition of the animal using a trained neural network; and
outputting a notification based on the classification indication to a computing device associated with at least one of the facility or a buyer, the notification indicating at least one of the following: an indication of the body composition of the subject; an indication of the gait quality of the subject; a productivity prediction for the subject; or a recommended intervention for the subject.

2. The method of claim 1, wherein the category of the plurality of categories is determined based on a score between a continuous range of scores.

3. The method of claim 1, wherein the category of the plurality of categories is determined based on previously determined categories on at least one of previous topologies, shapes, gaits, or body compositions.

4. The method of claim 2, wherein the category of the plurality of categories is further determined based on a threshold to compare the at least one of the topology of the animal, the shape of the animal, the gait of the animal, or the body composition of the animal with the threshold.

5. The method of claim 1, wherein the gait of the animal is determined by:
identify a joint in a first frame of the number of video frames with a mark;
porting the identified joint in the first frame to a second frame of the number of video frames;
determining a time-series relative motion of the joint based on the joint in the first frame and the joint in the second frame; and
determining the gait of the animal based on the time-series relative motion.

6. The method of claim 5, wherein the gait of the animal is provided to the neural network trained to identify categories of the gait, and
wherein the neural network was trained on a dataset comprising previous animal gait information and the categories in connection of the previous animal gait information.

7. The method of claim 1, further comprising: determining an indicator of the animal's backfat by measuring a region of the animal from the video data.

8. The method of claim 1, further comprising: determining an indicator of the body composition of the animal by determining at least one of a height, shoulder width, estimated weight, and estimated volume of the animal from the video data.

9. A precision livestock farming system comprising:
a camera; and
a processor,
wherein the precision livestock farming system is further characterized by a memory in communication with the processor, having stored thereon a set of instructions which, when executed, cause the processor to:
acquire data regarding an animal of interest from the camera during a given time period;
determine at least one of a body composition indicator or a pose indicator based on the data acquired from the camera;
store the body composition indicator or pose indicator in a data record associated with the animal of interest; and
provide the body composition indicator or pose indicator to a neural network trained to predict an animal outcome for animals of a similar species to the animal of interest.

10. The system of claim 9, wherein the camera is a depth camera.

11. The system of claim 10, wherein determining at least one of a body composition indicator or a pose indicator comprises determining landmarks of interest in a depth image of the animal of interest.

12. The system of claim 11, wherein determining landmarks of interest in the depth image further comprises using a landmark detector to identify landmarks of interest in another image of the animal of interest and transposing the landmarks of interest to the depth image.

13. The system of claim 9, wherein the neural network is trained to predict whether the animal of interest will exhibit an abnormal gait based upon a timeseries of depth image frames of a video clip of the animal of interest.

14. The system of claim 9, wherein the processor is further caused to output a notification to the farming facility identifying a health issue for the animal of interest based upon the output of the neural network.

15. The system of claim 9, wherein:
the camera is a near-infrared depth camera positioned within farming facility;
the processor is further caused to:
determine a gait abnormality for a batch of animals from a set of depth video clips of batch of animals acquired by the camera;
determine body composition scores of the batch of animals based upon at least one of a height, shape, backfat width, or volume of each animal of the batch of animals;
output the gait abnormality and body composition determinations to at least one of a network associated with the farming facility or a network associated with potential buyers of the batch of animals.
